(19) United States
(12) Patent Application Publication
(10) Pub. No.: US 2023/0248266 A1
(43) Pub. Date: Aug. 10, 2023

(54) CENTER OF GRAVITY CONTROL SUPPORT DEVICE AND METHOD

(71) Applicant: Nippon Telegraph and Telephone Corporation, Tokyo (JP)

(72) Inventors: Arinobu NIIJIMA, Musashino-shi, Tokyo (JP); Toki TAKEDA, Musashino-shi, Tokyo (JP); Takashi SATO, Musashino-shi, Tokyo (JP)

(21) Appl. No.: 18/014,429

(22) PCT Filed: Jul. 7, 2020
(86) PCT No.: PCT/JP2020/026567

§ 371 Date: Jan. 4, 2023

(51) Int. Cl. A61B 5/11; A61B 5/00

(52) U.S. Cl. CPC A61B 5/1116; A61B 5/7455

(57) ABSTRACT

The present invention includes: a center of gravity measurement unit 10 configured to measure a change in a center of gravity position of a standing posture of a user; a tactile stimulation signal generation unit 20 configured to, in a case where the center of gravity position deviates from a predetermined range, generate a tactile stimulation signal for applying tactile stimulation to the user; and a tactile stimulation presentation unit 30 configured to convert the tactile stimulation signal into tactile stimulation and present the tactile stimulation to a sole of a foot of the user.

### TECHNICAL FIELD

The present invention relates to a center of gravity control support device and a method thereof.

### BACKGROUND ART

In some cases, a person works in a standing posture by using a standing desk for the purpose of eliminating lack of exercise in desk work. The standing posture imposes an extra burden on the pelvis and muscles when, for example, the person works for a long time in a slouching posture. Deviation of the center of gravity of a body causes distortion of the body.

Therefore, in a case where the person works for a long time in the standing posture, it is preferable that there be a method of supporting the person to take a standing posture that does not impose a burden on the body. The standing posture can be detected by measuring a center of gravity position of the posture.

There is a method using a commercially available force plate, pressure sensor, or the like to measure the center of gravity position of the standing posture. A method of feeding back a result measured by the sensor to a user is, for example, notifying the user by display on a screen, sound, vibration, or the like.

The method of notifying the user by display or sound may block visual and auditory sensations of the user, which may interfere with the work. The method of notifying the user by vibration requires the user to consciously change the posture in response to the stimulation, which imposes a cognitive load on the user.

As a method that does not block the visual and auditory sensations or cause the cognitive load, there is a method of directly controlling a posture of a body by electrical stimulation (e.g. Non Patent Literature 1).

### CITATION LIST

### Non Patent Literature

Non Patent Literature 1: FUJITA Kinya and three others, “Feedback control method for paraplegic standing by functional electrical stimulation”, The transactions of the Institute of Electronics, Information and Communication Engineers, D 75.1 (1992): 791-798

### SUMMARY OF INVENTION

### Technical Problem

However, the method disclosed in Non Patent Literature 1 has a problem that it is necessary to attach a plurality of electrodes to the body, which is bothersome and troublesome.

The present invention has been made in view of the problem, and an object of the present invention is to provide a center of gravity control support device and a method thereof, each of which does not require attachment of electrodes to a body and is therefore not bothersome or troublesome.

### Solution to Problem

A center of gravity control support device according to an aspect of the present invention includes: a center of gravity measurement unit configured to measure a change in a center of gravity position of a standing posture of a user; a tactile stimulation signal generation unit configured to, in a case where the center of gravity position deviates from a predetermined range, generate a tactile stimulation signal for applying tactile stimulation to the user in accordance with a direction that deviates from the predetermined range; and a tactile stimulation presentation unit configured to convert the tactile stimulation signal into tactile stimulation and present the tactile stimulation to a sole of a foot of the user.

Further, a center of gravity control support method according to an aspect of the present invention, which is a center of gravity control support method performed by the above center of gravity control support device, includes: a center of gravity measurement step of measuring a change in a center of gravity position of a standing posture of a user; a tactile stimulation signal generation step of, in a case where the center of gravity position deviates from a predetermined range, generating a tactile stimulation signal for applying tactile stimulation to the user in accordance with a direction that deviates from the predetermined range; and a tactile stimulation presentation step of converting the tactile stimulation signal into tactile stimulation and presenting the tactile stimulation to a sole of a foot of the user.

### Advantageous Effects of Invention

According to the present invention, it is possible to perform center of gravity control that does not require attachment of electrodes to a body and is therefore not bothersome or troublesome.

### BRIEF DESCRIPTION OF DRAWINGS

FIG. 1 is a block diagram illustrating a functional configuration example of a center of gravity control support device according to an embodiment of the present invention.

FIG. 2 is a schematic diagram illustrating a state in which a user is using the center of gravity control support device of FIG. 1, which is viewed from the front.

FIG. 3 schematically illustrates an upper surface of the center of gravity control support device of FIG. 1.

FIG. 4 is a block diagram illustrating a more specific configuration example of FIG. 1.

FIG. 5 is a flowchart showing a processing procedure of a tactile stimulation signal generation unit and a tactile stimulation presentation unit of FIG. 1.

FIG. 6 illustrates a specific example of a tactile stimulation presentation unit.

FIG. 7 is a flowchart showing a processing procedure of a center of gravity control support method according to an embodiment of the present invention.

### DESCRIPTION OF EMBODIMENTS

Hereinafter, an embodiment of the present invention will be described with reference to the drawings. The same reference signs are given to the same components in a plurality of drawings, and description thereof will not be repeated.

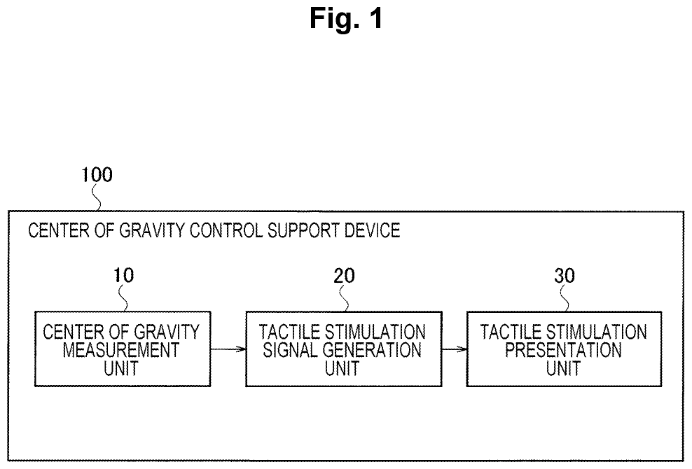

FIG. 1 is a block diagram illustrating a functional configuration example of a center of gravity control support device 100 according to an embodiment of the present invention. The center of gravity control support device 100 of FIG. 1 detects a center of gravity position of a standing posture of a user and, in a case where the center of gravity position deviates from a predetermined range, presents tactile stimulation to a sole of a foot of the user, thereby encouraging the user to improve the posture.

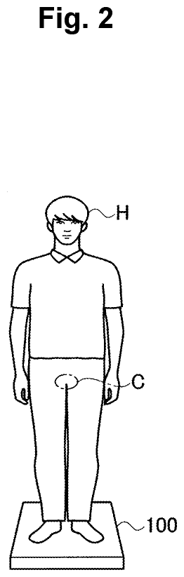

FIG. 2 is a schematic diagram illustrating a state in which a user is using the center of gravity control support device 100, which is viewed from the front. A user H uses the center of gravity control support device 100 while placing his/her both feet thereon like a scale.

A center of gravity C of the user H in the standing posture is at a position of his/her pelvis, and a line of gravity is a straight line passing through a center of the body. When viewed from the sides, two lines of gravity pass through ear holes, shoulders, hip joints, the back of knee joints, and the front of ankle joints of the user and lead to soles of both the feet.

When the user takes a standing posture that does not impose a burden on the body, the weight is equally applied to the soles of both the feet through the lines of gravity. Therefore, the center of gravity position can be detected as being located on a surface of the center of gravity control support device 100 immediately below the center of gravity C in a vertical direction. When the user takes an unbalanced posture that imposes a burden on the body, the center of gravity position shifts on the same plane from a position immediately below the center of gravity position.

The center of gravity control support device 100 according to the present embodiment detects the shift of the center of gravity position and, in a case where the center of gravity position deviates from a predetermined range, presents tactile stimulation to the user H and performs center of gravity control. The tactile stimulation stimulates a sensation of pain of a person and, for example, causes the flexion reflex.

The flexion reflex is, for example, a phenomenon that causes an action of unintentionally withdrawing his/her hand or foot when a person touches a hot object. The center of gravity control support device 100 controls the posture of the user by using, for example, the flexion reflex.

As illustrated in FIG. 1, the center of gravity control support device 100 includes a center of gravity measurement unit 10, a tactile stimulation signal generation unit 20, and a tactile stimulation presentation unit 30.

The center of gravity measurement unit 10 measures a change in the center of gravity position of the standing posture of the user H. The center of gravity measurement unit 10 measures the change in the center of gravity position by using, for example, a strain gauge. The strain gauge converts expansion and contraction caused by a load applied to a metal resistor into a change in resistance value.

In a case where the center of gravity position deviates from the predetermined range, the tactile stimulation signal generation unit 20 generates tactile stimulation for applying tactile stimulation to the user in accordance with a direction that deviates from the predetermined range.

The tactile stimulation presentation unit 30 converts a tactile stimulation signal into tactile stimulation and presents the tactile stimulation to a sole of a foot of the user H. For presenting the tactile stimulation, there is a method of applying hot stimulation at 45 degrees or more or cold stimulation at 15 degrees or less. There is also a method of bringing a protrusion into contact with the skin.

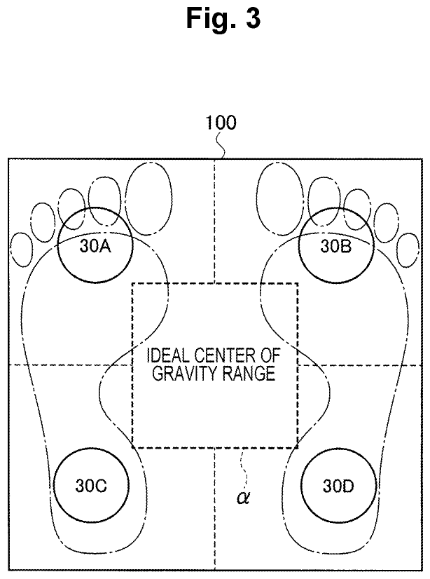

FIG. 3 schematically illustrates an upper surface of the center of gravity control support device 100. As illustrated in FIG. 3, for example, four tactile stimulation presentation units 30A, 30B, 30C, and 30D are arranged on the upper surface of the center of gravity control support device 100.

The tactile stimulation presentation unit 30A is arranged at a foot sole portion of a base of toes of his/her left foot, and the tactile stimulation presentation unit 30C is arranged at a foot sole portion of a heel of the left foot. The tactile stimulation presentation unit 30B is arranged at a foot sole portion of a base of toes of his/her right foot, and the tactile stimulation presentation unit 30D is arranged at a foot sole portion of a heel of the right foot.

In FIG. 3, a square range α surrounded by a broken line indicates a range of the center of gravity position in a case where the user H takes a well-balanced posture. The change in the center of gravity position of the user H is measured on the basis of whether or not the center of gravity position falls within the predetermined range α.

For example, in a case where the user H puts his/her weight on a front part of the left foot, the center of gravity position shifts toward the base of the toes of the left foot beyond the predetermined range α. When the shift of the center of gravity position is measured, the tactile stimulation signal generation unit 20 generates a tactile stimulation signal for applying tactile stimulation to the tactile stimulation presentation unit 30A. Then, the user H feels stimulation near the base of the toes of the left foot, reflexively stops the change of the posture that puts the weight on the front part of the left foot, and performs the flexion reflex to shift the weight in an opposite direction.

In a case where the user H puts the weight on the heels of both the feet, the center of gravity position shifts toward the center of a line connecting the heels of both the feet beyond the predetermined range α. When the shift of the center of gravity position is measured, the tactile stimulation signal generation unit 20 generates a tactile stimulation signal for applying tactile stimulation to the tactile stimulation presentation units 30C and 30D. Then, the user H feels stimulation near the heels of both the feet, reflexively stops the change of the posture that puts the weight on rear parts of both the feet, and performs the flexion reflex to shift the weight to the front parts of both the feet.

By generating tactile stimulation that stimulates, for example, the sensation of pain in a direction in which a load is applied as described above, it is possible to cause the flexion reflex and quickly return the center of gravity position of the user H within the predetermined range α.

As described above, the center of gravity control support device 100 according to the present embodiment includes: the center of gravity measurement unit 10 configured to measure the change in the center of gravity position of the standing posture of the user H; the tactile stimulation signal generation unit 20 configured to, in a case where the center of gravity position deviates from the predetermined range α, generate a tactile stimulation signal for applying tactile stimulation to the user H in accordance with a direction that deviates from the predetermined range; and the tactile stimulation presentation unit 30 configured to convert the tactile stimulation signal into tactile stimulation and present the tactile stimulation to the sole of the foot of the user H. This makes it possible to perform the center of gravity control that does not require attachment of electrodes to a body and is therefore not bothersome or troublesome.

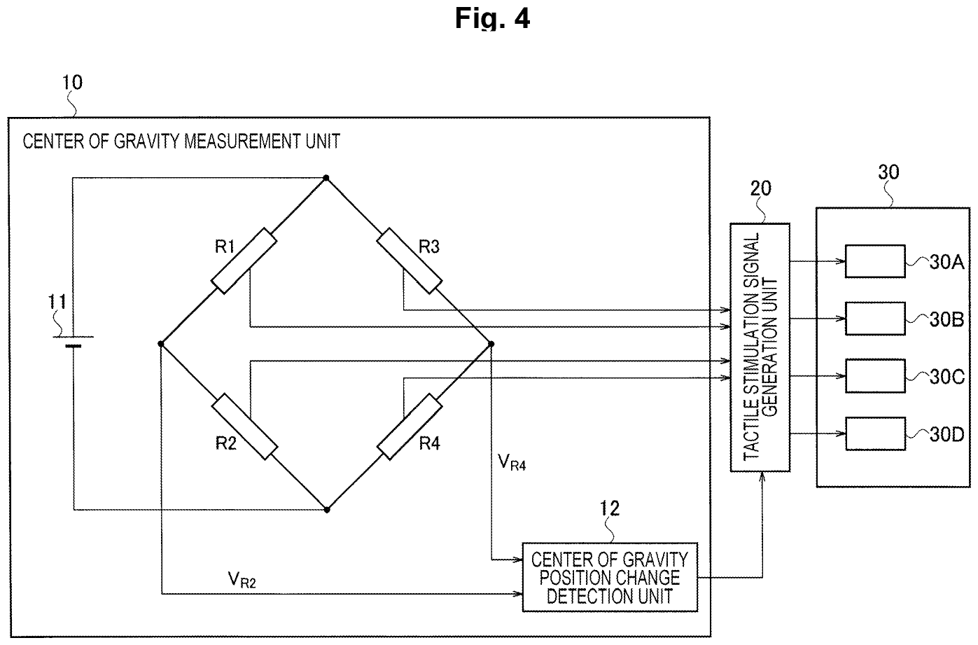

FIG. 4 is a block diagram illustrating a more specific configuration of the center of gravity control support device 100. Each unit will be described with reference to FIG. 4.

(Center of Gravity Measurement Unit)

The center of gravity measurement unit 10 includes a power supply 11, four strain gauges R1, R2, R3, and R4, and a center of gravity position change detection unit 12.

The power supply 11 applies a voltage to a bridge formed by the strain gauges R1, R2, R3, and R4. The power supply 11 may be a power supply of the center of gravity control support device 100.

The strain gauges R1 and R2 are connected in series and are connected in parallel to the strain gauges R3 and R4 similarly connected in series. The four strain gauges R1, R2, R3, and R4 form a bridge.

The four strain gauges R1, R2, R3, and R4 have the same resistance value and have the same rate of change in resistance value when a strain is applied. The strain gauges R1, R2, R3, and R4 are arranged near, for example, the tactile stimulation presentation units 30A, 30B, 30C, and 30D, respectively, in FIG. 3.

As illustrated in FIG. 3, the user H takes the standing posture while placing the left foot of the user H on the tactile stimulation presentation units 30A and 30C and placing the right foot of the user H on the tactile stimulation presentation units 30B and 30D. When the user takes a well-balanced standing posture that does not impose a burden on the body, the weight is equally applied to the soles of the feet through the lines of gravity passing through both the feet.

Then, the resistance value of each of the strain gauges R1, R2, R3, and R4 changes due to application of a load, but each of the strain gauges R1, R2, R3, and R4 changes in the same manner. Therefore, the bridge maintains a balanced state, and thus no potential difference is generated between a voltage V_{R2} at a connection point of the strain gauges R1 and R2 and a voltage VR₄ at a connection point of the strain gauges R3 and R4.

Meanwhile, when the user H takes an unbalanced posture that imposes a burden on the body, the bridge enters an unbalanced state, and a potential difference is generated between the voltage VR₂ and the voltage VR₄. When the potential difference exceeds a predetermined magnitude, the center of gravity position change detection unit 12 detects that the center of gravity position of the user H deviates from the predetermined range.

For example, the user H is assumed to shift the weight to the front part of the left foot. In this case, a cross-sectional area of the strain gauge R1 decreases and the resistance value increases. Alternatively, a length of a resistor of the strain gauge R1 may increase when a load is applied. In either case, the resistance values of the strain gauges R1 to R4 increase when a load is applied.

In a case where the user H shifts the weight to the front part of the left foot, the resistance value of the strain gauge R1 becomes the maximum. In a case where the user H shifts the weight to the rear part of the left foot, the resistance value of the strain gauge R2 becomes the maximum.

Similarly, in a case where the user H shifts the weight to the front part of the right foot, the resistance value of the strain gauge R3 becomes the maximum. In a case where the user H shifts the weight to the rear part of the right foot, the resistance value of the strain gauge R4 becomes the maximum. As described above, it is possible to measure the shift of the center of gravity position.

(Tactile Stimulation Signal Generation Unit)

In a case where the center of gravity position of the user H deviates from the predetermined range, the tactile stimulation signal generation unit 20 generates a tactile stimulation signal for applying tactile stimulation to the user H in accordance with a direction that deviates from the predetermined range. In the present embodiment, the tactile stimulation signal generation unit 20 generates a tactile stimulation signal corresponding to any of the strain gauges R1, R2, R3, and R4 indicating the maximum resistance value.

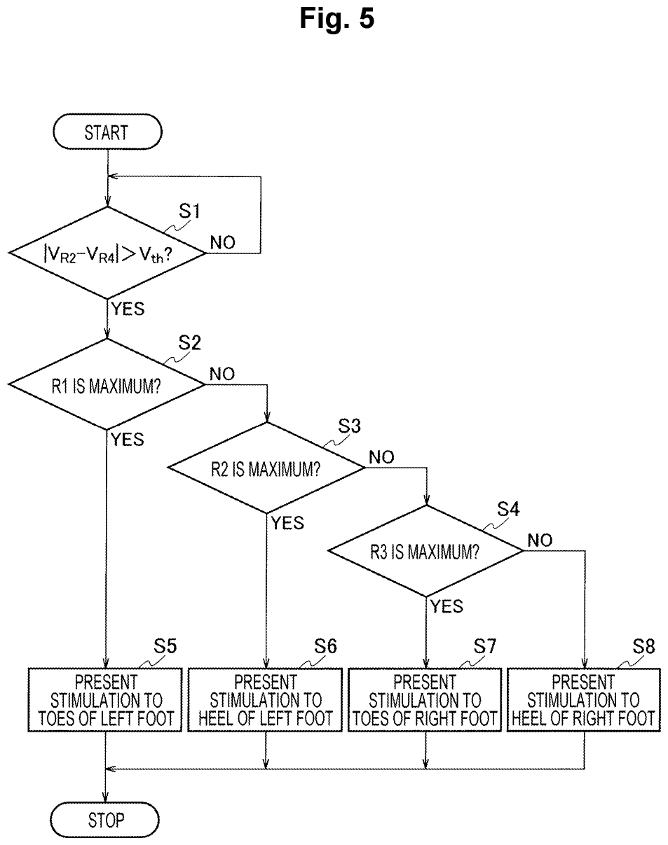

FIG. 5 is a flowchart showing a processing procedure of the center of gravity position change detection unit 12 and the tactile stimulation signal generation unit 20.

When the center of gravity position of the user H deviates from the predetermined range, the center of gravity position change detection unit 12 instructs the tactile stimulation signal generation unit 20 to generate a tactile stimulation signal (step S1). The instruction is issued in a case where the potential difference between the voltage V_{R2} and the voltage V_{R4} exceeds a predetermined threshold Vₜₕ.

In a case where the potential difference between the voltage V_{R2} and the voltage V_{R4} does not exceed the predetermined threshold Vₜₕ, it is unnecessary to encourage the user H to change the posture, and thus a process of detecting the change in the center of gravity position is repeated (NO in step S1).

When receiving input of a signal instructing the tactile stimulation signal generation unit 20 to generate a tactile stimulation signal from the center of gravity position change detection unit 12, the tactile stimulation signal generation unit 20 determines which one of the strain gauges R1 to R4 has the maximum resistance value (steps S2 to S4).

In a case where the resistance value of the strain gauge R1 is the maximum, a tactile stimulation signal for presenting tactile stimulation in the tactile stimulation presentation unit 30A arranged near the base of the toes of the left foot is generated (step S5).

In a case where the resistance value of the strain gauge R2 is the maximum, a tactile stimulation signal for presenting tactile stimulation in the tactile stimulation presentation unit 30C arranged near the heel of the left foot is generated (step S6).

In a case where the resistance value of the strain gauge R3 is the maximum, a tactile stimulation signal for presenting tactile stimulation in the tactile stimulation presentation unit 30B arranged near the base of the toes of the right foot is generated (step S7).

In a case where the resistance value of the strain gauge R4 is the maximum, a tactile stimulation signal for presenting tactile stimulation in the tactile stimulation presentation unit 30D arranged near the heel of the right foot is generated (step S8).

As described above, it is possible to generate a tactile stimulation signal corresponding to a shift direction of the center of gravity position of the user H.

(Tactile Stimulation Presentation Unit)

The tactile stimulation presentation unit 30 converts a tactile stimulation signal generated by the tactile stimulation signal generation unit 20 into tactile stimulation and presents the tactile stimulation to the sole of the foot of the user H. The tactile stimulation may be any of thermal stimulation, contact stimulation, vibration stimulation, electrical stimulation, ultrasonic stimulation, and wind stimulation.

In the present embodiment, an example where thermal stimulation is presented by a Peltier element will be described.

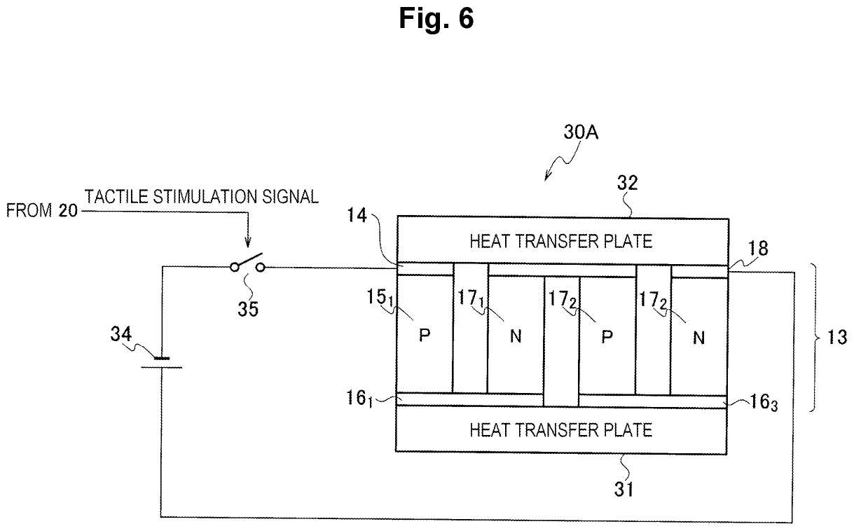

FIG. 6 schematically illustrates a configuration example where one tactile stimulation presentation unit 30A included in the tactile stimulation presentation unit 30 is formed as a Peltier element. The same applies to the other tactile stimulation presentation units 30B to 30D.

(Peltier Element)

The tactile stimulation presentation unit 30A (hereinafter, the Peltier element 30A) is formed by sandwiching a thermoelectric module 13 between two heat transfer plates 31 and 32 and transfers heat from one heat transfer plate 31 to the other heat transfer plate 32. The heat transfer plates 31 and 32 are made from, for example, a copper material having a relatively high thermal conductivity and being inexpensive.

The thermoelectric module 13 includes a positive electrode 14, p-type thermoelectric material 15₁ and 15₂, common electrodes 161, 162, and 163, n-type thermoelectric material 17₁ and 17₂, and a negative electrode 18. Each of the heat transfer plates 31 and 32 and each electrode (the positive electrode 14, the negative electrode 18, and the common electrodes 161, 162, and 163) are insulated by an insulating layer (not illustrated).

A negative electrode of the power supply 34 is connected to the positive electrode 14, and a positive electrode of the power supply 34 is connected to the negative electrode 18. Then, holes of the p-type thermoelectric material 15₁ and 15₂ move from the heat transfer plate 31 toward the heat transfer plate 32. Electrons of the n-type thermoelectric material 17₁ and 17₂ also move from the heat transfer plate 31 toward the heat transfer plate 32. Heat around the heat transfer plates 31 and 32 is carried by the holes and the electrons serving as carriers, and thus, in this example, the heat transfer plate 31 acts as a heat absorbing plate, and the heat transfer plate 32 acts as a heat radiating plate. Hereinafter, the heat transfer plates will also be referred to as the heat absorbing plate 31 and the heat radiating plate 32.

When a current is applied in an opposite direction, the holes and the electrons move in the opposite direction. Thus, the heat transfer plate 31 acts as a heat radiating plate and the heat transfer plate 32 acts as a heat absorbing plate. In the example of FIG. 6, in order to explain the principle of the Peltier element 30A, the n-type thermoelectric material 17 and the p-type thermoelectric material 15 are laminated only in two layers. In order to obtain practical actions of heat generation and heat absorption, the n-type thermoelectric material 17 and the p-type thermoelectric material 15 are laminated in multiple layers.

As described above, by bringing either one of the heat transfer plates 31 (32) of the Peltier element 30A that receives supply of a current to radiate and absorb heat into contact with the skin of a human body, it is possible to present either hot stimulation or cold stimulation.

The thermal stimulation is presented when a tactile stimulation signal generated by the tactile stimulation signal generation unit 20 turns on a switch 35. In order to stimulate a sensation of pain of a person, it is only necessary to stimulate skin of a sole of his/her foot at a temperature of 45 degrees or more in the hot stimulation and 15 degrees or less in the cold stimulation.

As a result of verification with a subject, it was confirmed that the flexion reflex certainly occurred when the thermal stimulation was presented at 60 degrees or more. The user H immediately changes the posture because of the flexion reflex.

(Center of Gravity Control Method)

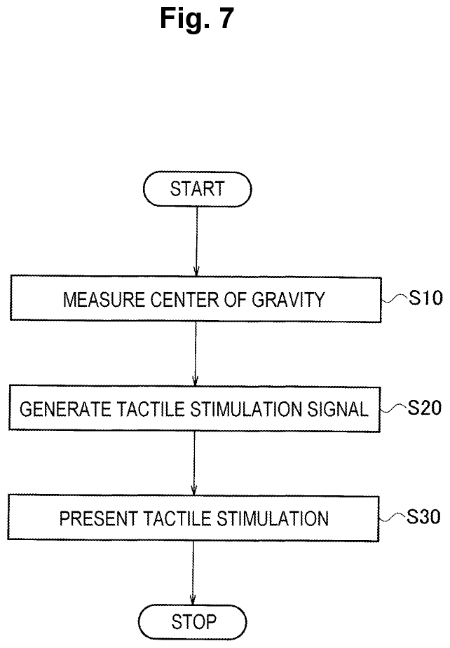

FIG. 7 is a flowchart showing a processing procedure of a center of gravity control support method performed by the center of gravity control support device 100 according to the present embodiment.

When the center of gravity control support device 100 starts operation, the center of gravity measurement unit 10 measures a change in the center of gravity position of the standing posture of the user H (step S10).

In a case where the center of gravity position of the user H deviates from the predetermined range, the tactile stimulation signal generation unit 20 generates a tactile stimulation signal for applying tactile stimulation to the user H in accordance with a direction that deviates from the predetermined range (step S20).

The tactile stimulation presentation unit 30 converts the tactile stimulation signal generated by the tactile stimulation signal generation unit 20 into tactile stimulation and presents the tactile stimulation to the sole of the foot of the user H (step S30).

As described above, the center of gravity control support method, which is a center of gravity support control method performed by the center of gravity control support device 100, includes: a center of gravity measurement step (S10) of measuring the change in the center of gravity position of the standing posture of the user H; a tactile stimulation signal generation step (S20) of, in a case where the center of gravity position deviates from the predetermined range, generating a tactile stimulation signal for applying tactile stimulation to the user H in accordance with a direction that deviates from the predetermined range; and a tactile stimulation presentation step (S30) of converting the tactile stimulation signal into tactile stimulation and presenting the tactile stimulation to the sole of the foot of the user H. This makes it possible to perform the center of gravity control that does not require attachment of electrodes to the body of the user H and is therefore not bothersome or troublesome.

As described above, according to the present invention, it is unnecessary to attach electrodes to the body of the user H. Therefore, it is possible to perform the center of gravity control that is not bothersome or troublesome.

In the measurement of the center of gravity in the above embodiment, the strain gauges form a bridge as an example. However, the present invention is not limited to this example. Resistance values of respective sensors may be measured without forming a bridge, and the change in the center of gravity position may be obtained on the basis of the resistance values. The sensors are not limited to the strain gauges.

A plurality of sensors, which is equal to or more than four, for detecting the center of gravity position may be provided. The change in the center of gravity position may be detected in multiple directions equal to or more than four directions, and tactile stimulation may be presented in accordance with the directions. The center of gravity position may also be measured by capturing an image of the user by using a camera and performing image analysis.

Although the tactile stimulation has been exemplified by the thermal stimulation, for example, contact stimulation using a solenoid pin may be presented. Further, the tactile stimulation has been exemplified by an example where a sensation of pain of a person is stimulated to cause the flexion reflex. However, the present invention is not limited to this example. Tactile stimulation that does not cause the flexion reflex may be presented.

Thus, it is needless to say that the present invention includes various embodiments and the like not described herein. Therefore, the technical scope of the present invention is defined only by the matters specifying the invention according to the claims appropriate from the above description.

### REFERENCE SIGNS LIST

10 center of gravity measurement unit
12 center of gravity position change detection unit
20 tactile stimulation signal generation unit
30, 30A, 30B, 30C, 30D tactile stimulation presentation unit
100 center of gravity control support device

1. A center of gravity control support device comprising:
a center of gravity measurement unit, including one or more processors, configured to measure a change in a center of gravity position of a standing posture of a user;
a tactile stimulation signal generation unit, including one or more processors, configured to, in a case where the center of gravity position deviates from a predetermined range, generate a tactile stimulation signal for applying tactile stimulation to the user in accordance with a direction that deviates from the predetermined range; and
a tactile stimulation presentation unit, including one or more processors, configured to convert the tactile stimulation signal into tactile stimulation and present the tactile stimulation to a sole of a foot of the user.

2. The center of gravity control support device according to claim 1, wherein
the tactile stimulation is thermal stimulation.

3. A center of gravity control support method performed by a center of gravity control support device, the method comprising:
a center of gravity measurement step of measuring a change in a center of gravity position of a standing posture of a user;
a tactile stimulation signal generation step of, in a case where the center of gravity position deviates from a predetermined range, generating a tactile stimulation signal for applying tactile stimulation to the user in accordance with a direction that deviates from the predetermined range; and
a tactile stimulation presentation step of converting the tactile stimulation signal into tactile stimulation and presenting the tactile stimulation to a sole of a foot of the user.

4. The center of gravity control support method according to claim 3, wherein the tactile stimulation is thermal stimulation.
